(19) United States
(12) Patent Application Publication
(10) Pub. No.: US 2015/0025025 A1
(43) Pub. Date: Jan. 22, 2015

(54) Immuno-modulators for treating functional epithelial syndromes

(71) Applicant: Ricky McCullough, Foster, RI (US)

(72) Inventor: Ricky McCullough, Foster, RI (US)

(21) Appl. No.: 13/946,260

(22) Filed: Jul. 19, 2013

(51) Int. Cl. A61K 31/733; A61K 31/702; A61K 31/7048; A61K 31/65

(52) U.S. Cl. CPC A61K 31/733; A61K 31/65; A61K 31/702; A61K 31/7048
USPC 514/25; 514/152; 514/54

(57) ABSTRACT

An immuno-modulating composition including an organic compound and a metal chelator or a metal ion. Also disclosed is a method for treating a disease or condition in man and animals with the composition. Further, a method for delivering a growth factor or a homeostasis-maintaining factor to its receptor is provided.

### BACKGROUND

The problem at times in mammalian diseases is that for some reason or another, growth factors do not govern as effectively as they are capable of doing. When this occurs, a disease state persists longer than need be or regular homeostatic processes degenerate into a disease state and tissue dysfunction.

All tissue processes occur in phases, and in certain processes the presence and action of a growth factor is critical to the movement of the process toward its end goal. In mammalian tissues, heparin like substances are used to chaperone locally elaborated growth factors to tyrosine kinase receptor sites. One type of immuno-modulator is a substance that, when applied pharmaceutically, will chaperone the phase-critical growth factors to their respective receptor sites, activating the sites in a time-efficient manner. An effective immuno-modulator that mimics the action of mammalian heparin-like substances would be an obvious solution. But true heparin-like substances in large doses act as anticoagulants, and will lead to bleeding.

It is known that concentrations of various polyanion compounds can non-specifically chaperone growth factors to their cognate tyrosine kinase receptors to activate the receptors. Their binding while measurably present is impractical to use as a pharmaceutical in the treatment of diseases. An appropriately structured immuno-modulator can affect the expressed balance of cytokines present within a living viable mammalian tissue in the direction of a disease amelioration or homeostasis affirmation.

Pro-inflammatory cytokines and anti-inflammatory cytokines are in states of flux with imbalances that are advantageous for all tissues to maintain a condition of homeostasis. In the process of maintaining a state of homeostasis, there are advantages to having an imbalance of pro vs anti inflammatory cytokines To remove dying cells, noxious materials, toxins, or simply to heal, mammalian tissues use timed and sequenced imbalances of pro-versus anti-inflammatory cytokines to accomplish, in stepwise fashion, vital micro-processes to restore homeostasis following an assault or to maintain homeostasis in the course of replacing senescent cells with new regenerated ones.

The ebb and flow of these imbalances are genetically orchestrated by a broad class of tyrosine kinase receptor activators known as growth factors.

Membrane-bound tyrosine kinase receptor activation is associated with a wide array of physiological processes, for example, tissue repair, cell growth, embryogenesis, angiogenesis, differentiation, and wound healing. The class of substances that activate these receptors in a timed and orchestrated manner is known as growth factors.

Growth factors are made constitutively. Their presence and effect can be augmented by increased production (which usually is very slight) and by increased membrane expression of tyrosine kinase receptors (which appears the more preferred choice of augmentation by mammalian tissues).

Growth factors are present on site throughout the mucosal lining. They can be elaborated by all epithelial cells, fibrocytes, as well as by neurons. They can be manufactured on the spot by portable circulating cells, e.g., platelets, monocytes, and mast cells etc. And in every case growth factors carry out nearly one single function, the activation of membrane bound tyrosine-kinase receptors.

These receptors are genetically induced to be expressed in the membranes of cells within a tissue and once activated these receptors will orchestrate the process required for the tissue to either re-establish or re-affirm the continuation of homeostasis.

The molecules responsible for carrying out the required processes are the pro- and anti-inflammatory cytokines The “ebb and flow imbalance” between them is genetically determined by the process-specific signaling required for the task at hand, e.g., tissue repair and wound healing.

The role of growth factors is to apply “efficiency and orderliness” to the chaotic process conducted by the cytokines induced by genetic signals within the cell nucleus, the genetic signals themselves arising as a result of stimuli emanating from the cellular membrane. Once that stimuli halts or changes, the genetic signals correspondingly halt or change.

The orderly and efficient governance by growth factors is effected by their engagement of newly expressed membrane-bound tyrosine kinase receptors. The properly timed presence of growth factors and the timed induction of their membrane-bound tyrosine kinase receptors there is an efficient and orderly balance between the aggressive/destructive actions of the pro-inflammatory cytokines and regressive/slowing moderating actions of the anti-inflammatory cytokines

For example, Werner and Grose (Werner S, Grose R: Regulation of wound healing by growth factors and cytokinesPhysiol Rev 83:835-870, 2003) outlined the significance of growth factors in governance of healing of acute wounds. They identified three distinct stages of healing gaping wounds that span two to three weeks. First, the wound is filled with a blood clot which is infiltrated by neutrophils, occuring in 12-24 hours. Second, the population of neutrophils gives way to an abundance of infiltrating macropahges, which cause a migration of endothelial cells to form new blood vessels between days 3-7 following injury. The clot dries and begins to harden, initially at its superior aspect and progresses inferiorly, while granulation tissue develops below the clot at its inferior aspect. During this time fibroblasts migrate into the wound particularly into the granulation tissue causing the epithelial cells at the remote edges of the wound to migrate toward each other undermining the drying clot (hastening its solidification into a scab) and separating it from the softer granulation tissue beneath. In the third stage, which takes place 1-2 weeks after injury, the wound, filled with granulation tissue, is infiltrated by more fibroblasts, with older fibroblasts transforming into myofibroblasts to contract the wound and deposit collagen.

Each stage of acute wound healing described above involves the genetically orchestrated use of pro-inflammatory and anti-inflammatory cytokine signals, which in turn are governed by the presence of growth factors, the expression of growth factor receptors on target cells, and the rate of action applied by growth factor on those cells.

Every organ system in which the epithelium—endothelium, epidermis, or mucosal surfaces—relies on the genetically controlled interplay of pro-inflammatory and anti-inflammatory cytokines to maintain homeostatic function (which includes acute repair as well as “wear & tear” replacement) is vulnerable to some dysfunction, which can be governed effectively or controlled ineffectively. Ineffective control leads to the development of functional epithelial syndromes and organ dysfunction.

While others have recognized that cytokine imbalance exists and that either selective immuno-modulation or direct manipulation of cytokines may possibly remedy these disease states, few practical solutions have been presented and fewer still have operated effectively to be of substantial benefit.

There is a need to develop pharmaceutical compositions that can be used to successfully treat diseases and functional epithelial syndromes that result from a cytokine imbalance.

### SUMMARY

The main objective of the present invention is to provide a pharmaceutically active immuno-modulator. Another objective is to provide a method for treating diseases that result from cytokine imbalances by administering the pharmaceutically active immuno-modulator. Thus, one aspect of this invention relates to an immuno-modulating composition for engaging a homeostasis-maintaining factor. The composition includes an organic compound, a metal ion, and a metal chelator. The valency molar ratio between the organic compound, the metal ion, and the metal chelator is 1:1:1 to 10:1:1 and the organic compound contains polar constituents covalently attached such that the polar constituents have a minimally restrictive stereographic ability of rotation. In a particular embodiment, the immuno-modulating composition lacks the metal ion and the valency molar ratio between the organic compound and the metal chelator is 1:1 to 10:1. In yet another embodiment, the immuno-modulating composition lacks the metal chelator and the valency molar ratio between the organic compound and the metal ion is 1:1 to 10:1.

Another aspect of this invention relates to a method for treating a disease or condition with a therapeutically effective amount of a pharmaceutically active immuno-modulator. The immuno-modulator includes an organic compound, a metal ion, and a metal chelator. The valency molar ratio between the organic compound, the metal ion, and the metal chelator is 1:1:1 to 10:1:1 and the organic compound is a fatty acid, a gum, a starch, a polyhydrol, a sulfonate, a phosphonate, a heterocyclic compound, a multi-cyclic compound, a carboxylic acid, or a carbamyl compound. In alternative embodiments, the immuno-modulator lacks the metal ion or lacks the metal chelator.

Still another aspect of this invention relates to a method for treating a gastrointestinal disorder in a monogastric or polygastric animal by administering a pharmaceutically active immuno-modulator that includes an organic compound, a metal ion, and a metal chelator. The valency molar ratio between the organic compound, the metal ion, and the metal chelator is 1:1:1 to 10:1:1 and the organic compound contains polar constituents covalently attached such that the polar constituents have a minimally restrictive stereographic ability of rotation. In specific embodiments, the metal ion or the metal chelator is omitted.

Also provided is a method for converting an organic molecule into a pharmaceutically active immuno-modulator, including the steps of mixing the organic molecule with a metal chelator to form a solution and adding a multivalent metal cation to the solution to form a complex between the carrier, the multivalent metal ion, and the metal chelator.

Additionally, a method for delivering a growth factor or a homeostasis-maintaining factor to its receptor is provided. The method is carried out by combining the growth factor or the homeostasis-maintaining factor with a composition that includes a polyionic carrier, a metal ion, and a metal chelator and contacting the combined growth factor or homeostasis-maintaining factor with a target cell containing a receptor for the growth factor or homeostasis-maintaining factor. The polyionic carrier is selected from the group consisting of a fatty acid, a gum, a tarch, a polyhydrol, a sulfonate, a phosphonate, a heterocyclic compound, a multi-cyclic compound, a carboxylic acid, or a carbamyl compound. The composition accelerates binding of the growth factor or homeostasis-maintaining factor to the receptor on the target cell and activation of the receptor as compared to the growth factor or homeostasis-maintaining factor alone.

The details of one or more embodiments of the invention are set forth in the description below. Other features, objects, and advantages of the invention will be apparent from the description and from the claims.

### DETAILED DESCRIPTION

It is disclosed that broad classes of carbonaceous organic compounds can be converted into immuno-modulators. The immuno-modulating substances accelerate restoration of cytokine balance and thereby favorably alter the course of particular disease states.

A carbonaceous organic compound can be converted into an immuno-modulator by suspension and dissolution in a solvent of a suitable carbonaceous organic compound having predominantly negative valency substituents together with a cation and an avid cation chelator. A carbonaceous organic compound with mostly positive valency substituents can be converted into an immune-modulator by suspending and dissolving it in a solvent together with an anion and an anion chelator.

The carbonaceous organic compounds can be naturally occurring ones such as tannins, resveratrol, mucilages, gums, bile acids, and starches, as well as their chemically altered derivatives. Other compounds that can be used include tetracycline, doxycycline, poly sulfates, poly phosphonates, fluoroquinolones, benzydamine, cefditoren pivoxil, rifaximin, benzydamine, cefuroxime axetil, ezetimibe, and compounds similar to these in nature. The carbonaceous organic compounds can contain polar substituents covalently attached such that they have a minimally restrictive stereographic ability of rotation. Polar substituents can include bi, tri-, quadric-oxygenated metallic elements such as phosphates, sulfates, and nitrates.

In one embodiment, the carbonaceous organic compound is combined with multi-dentate chelators and multi-valent ions. In an alternate embodiment, uni-dentate chelators and uni-valent ions can be used.

Multi-dentate chelators that can be used include carboxylic acids (e.g., EDTA, DPTA, malate, oxalate, citrate), multi-phenyl compounds, organic diamines (e.g., EDA, BDA, DTA, MDTA, TEPA, DAP, PDA, HDA, IBPA, MIBPA, BPETA, DMPDA, and PIP) and polyphosphonates such as methlylene diphosphonate, hydroxyl ethylene diphosphonates, and amino ethylene diphosphonates. Transitional metal oxides, fluorides, chlorides, or sulfides can act as multi-dentate chelators as well, particularly if the carbonaceous compound carrier is heterocyclic, such as tetracycline or doxycycline.

Uni-dentate chelators that can be used include, but are not limited to, mesalamine; short chain fatty acids such as butyric, proprionic, acetic, and formic acids; and gabapenitin.

Multivalent metal ions that can be used include those elements that have a single charge, preferably more than one, attributable to its atomic polarity. The charge polarity of the metal ion should be opposite that of the ionic charge of the chelator and carbonaceous compound.

A suitable cation chelator ideally should have a binding capacity closely equivalent to the valence charge of the cation which it will chelate.

The ratio of admixture depends upon the polarity of the substituents on the carbonaceous organic compound and the association avidities of the cation and its cation chelator or the anion and its anion chelator.

In general the carbonaceous organic compound should be in a 1:1:1 to 10:1:1 admixture ratio of carbonaceous organic compound: chelator: cation or anion, determined by the molar contribution of the valency charge of the substitutents compared to the valency charge of the cation or anion.

For example if the carbonaceous organic compound contains 1 substituent with an equivalent negative polarity valency charge approximating 1, then the cation valency charge should also approximate 1 in the opposite polarity and its chelator should should have a negative valency charge that does not exceed negative 1.

Multivalent cations (2⁺, 3⁺) are preferred and should be matched with multi-dentate chelators containing at least 2 areas of chelation equidistantly spaced from each other and capable of chelating 1 cation. To this cation and chelator mixture is added a carbonaceous organic compound containing 1 to 4 or more electronegative substituents spaced evenly throughout the compound.

The spatial appointment of substituents throughout the compound is matched by the areas of dentition spatially distributed along the axis of the cationic chelator. The conversion reaction occurs at the instant of the addition of a multivalent cation to a solution containing the carbonaceous compound and the chelator, the latter two components characterized, respectively, by a favorable distribution of electronegative substituents (throughout the carbonaceous compound) and the chelating dentition (across the length of the cation-avid multi-dentate chelator).

Larger carbonaceous organic compounds require more cation/cation chelator mixed with it to accommodate the increased number of polar substituents within the compound. The amount of cation/cation chelator used is sufficient to permit the formation of an incomplete (non-closing) clathrate-type of structure suspended in solution. The non-closing clathrate-type structure containing cation, cation chelator, and substituent-studded carbonaceous compound will reversibly form a clathrate-type super-structure that can efficiently carry a growth factor (better than the carbonaceous compound alone), thereby driving the growth factor to its receptor site where the growth factor will effect a more smooth, orderly metabolic tissue process within the mucosa, more smooth and orderly than otherwise possible without the above-described compositions.

In an alternative embodiment, the carbonaceous organic compound is in a 1:1 to 10:1 admixture ratio of carbonaceous organic compound to chelator and the cation or anion is omitted. In another embodiment, the admixture can contain the carbonaceous organic compound and the cation or anion in a 1:1 to 10:1 ratio and lacks the chelator.

In one embodiment, the compositions of this invention can be administered as a treatment for patients with a variety of ailments that are governed by effectively controlled or ineffectively controlled tissue cytokine imbalance. When effectively controlled cytokine imbalance is a means to restore homeostasis then such processes or ailments are considered “repair”. When ineffectively controlled cytokine imbalance fails to restore homeostasis and proceed as a state of chronic defense or chronic repair then this persistence lead to tissue dysfunction giving rise to well-defined disorders or disease causing morbidity and suffering. Ailments are either healed, with periods of repair wherein homeostasis is completely restored, or persist, with periods of chronic dysfunction wherein homeostasis is never completely restored. Each is an ailment wherein cytokine imbalance drives the process.

Treatment according to this invention of either type of ailment results in either the acceleration of the healing process which restores homeostasis sooner than otherwise possible or the complete amelioration of chronic tissue and organ dysfunction, restoring homeostasis to conditions defined by failure to return to normal function. For example, in clinical remission of inflammatory bowel disease, gut function appears grossly intact, while cytokine imbalance persists.

Ailments in which cytokine imbalance plays a significant role can be treated by administering the compositions described above. These ailments, also known as functional epithelial syndromes, include but are not limited to epithelial lacerations, wounds, burns and ulcerations, chemo-radiation oral ulcerations, oral mucositis, alimentary mucositis, gastroesophageal reflux disorder (GERD) or reflux esophagitis, Barrett's esophagitis, functional heartburn or NERD (non-erosive reflux disorder), gastritis, gastroenteritis, enteritis, enterocolitis, functional dyspepsia, celiac sprue, collagenous or lymphocytic colitis, chemo-radiation enteritis, Crohn's disease, irritable bowel syndrome (all forms), chemo-radiation colitis, ulcerative colitis, minimal colitis, and atherosclerosis.

Other inflammatory disorders treatable by administering the compositions described above are defined by domination by cytokine imbalance. These include but are not limited to chronic hepatitis, muscle strains, myositis, tendoligamentous strains, tendonitis, fibrositis, fibrosis, myotendonitis disorders like fibromyalia, osseous injuries, post-surgical states, mechanical post-traumatic states, cystitis (urinary or biliary), prostatitis, prostatic hypertrophy, orchitis, pelvic inflammatory disease, scours, enterocolitis, parvoenteritis, hemorrhagic gastroenteritis, veterinary peptic ulcer, gastritis, enteritis, or colitis.

Additionally, the above-described compositions can be used to treat disorders in livestock, such as gastrointestinal mucosal disorders in pigs, cows, horses, sheep, goats, and wild animals.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLES

### Example 1

### An Immuno-Modulating Suspension of Tetracycline

| Ingredients | Amount |
|---|---|
| tetracycline | 1,000 mg |
| malate or citrate | 1,000 mg |
| calcium carbonate or calcium chloride | 1,000 mg |
| water | 15 ml |

### Example 2

### An Immuno-Modulating Suspension of Inulin

| Ingredients | Amount |
|---|---|
| inulin | 4,000 mg |
| malate or citrate | 1,000 mg |
| calcium carbonate | 1,000 mg |
| water | 15 ml |

### Example 3

### An Immuno-Modulating Suspension of Fructo-Oligosaccharide

| Ingredients | Amount |
|---|---|
| fructo-oligosaccharide | 4,000 mg |
| malate or citrate | 1,000 mg |
| calcium carbonate or calcium sulfate | 1,000 mg |
| water | 15 ml |

### Example 4

### An Immuno-Modulating Suspension of Polysulfate Disaccharide

| Ingredients | Amount |
|---|---|
| sucralfate | 1,000 mg |
| malate or citrate | 250 mg |
| calcium carbonate or calcium sulfate | 250 mg |
| water | 10 ml |

### Example 5

### An Immuno-Modulating Suspension of Polygalacturonic-Rhamnose

| Ingredients | Amount |
|---|---|
| Elm USP | 1,000 mg |
| malate or citrate | 250 mg |
| calcium carbonate or calcium sulfate | 250 mg |
| water | 10 ml |

### Example 6

### An Immuno-Modulating Polysulfate Disaccharide Powder

| Ingredients | Amount per capsule |
|---|---|
| dehydrated immuno-modulating | 450 mg |
| polysulfate disaccharide suspension | |
| of Example 4 | |
| pectin | 400 mg |
| xanthan gum | 200 mg |
| calcium sulfate | 100 mg |

### Example 7

### An Immuno-Modulating Polygalacturonic-Rhamnose Powder

| Ingredients | Amount per capsule |
|---|---|
| dehydrated immuno-modulating | 450 mg |
| polygalacturonic-rhamnose | |
| suspension of Example 5 | |
| pectin | 400 mg |
| xanthan gum | 200 mg |
| calcium sulfate | 100 mg |

### Example 8

### Treatment of Lacerations with Immuno-Modulating Tetracycline, Inulin, and Fructo-Oligosaccharde Suspensions

Three embodiments of this invention were tested against saline placebo to assess the healing rate of experimental epithelial wounds in the forearm of a 58 year old male volunteer.

The tested embodiments included three carbonaceous organic compounds, i.e., tetracycline, inulin and fructo-oligosaccharide, combined with a dicarboxylic acid chelator (malate) and a divalent cation (calcium) in a 1:1:1 ratio for tetracycline, a 4:1:1 ratio for inulin, and a 4:1:1 ratio for fructo-oligosaccharide. Four superficial wounds of equal length and depth were created on the volar surface of the forearms of the volunteer in the following manner. Following cleansing of the skin with isopropyl alcohol and the injection of 1 ml of 2% lidocaine subcutaneously, a sterile No.11 blade was used to incise the skin, creating 4 equal wounds measuring 6 mm long, 2 mm deep with a gaping width of 3 mm. Spontaneous bleeding slowed in 4 minutes and stopped completely by 7 minutes.

A sterile sodium chloride solution was applied to Wound 1 using a cotton-tipped applicator. The immuno-modulating fructo-oligosaccharide suspension described above in Example 3 was applied to Wound 2, the immuno-modulating inulin suspension of Example 2 was applied to Wound 3, and the immuno-modulating tetracycline suspension of Example 1 was applied to Wound 4.

Healing of the wounds was monitored for 7 days. A summary of the results is presented in the table below.

TABLE 1
7 Day Wound Healing in 58 y/o Volunteer

| Time | Wound 1 | Wound 2 | Wound 3 | Wound 4 |
|---|---|---|---|---|
| | Saline (Placebo) | Immuno-modulating | Immuno-modulating | Immuno-modulating |
| | | Fructo-oligosacchardide | Inulin | Tetracycline |
| | | (Example 3 above) | (Example 2 above) | (Example 1 above) |
| Time 0 | Wound Created | Wound Created | Wound Created | Wound Created |
| Time 4 minutes | Bleeding Slowed | Bleeding Slowed | Bleeding Slowed | Bleeding Slowed |
| Time 7 minutes | Bleeding Stopped | Bleeding Stopped | Bleeding Stopped | Bleeding Stopped |
| Time 8 minutes | Treatment & Adhesive | Treatment & Adhesive | Treatment & Adhesive | Treatment & Adhesive |
| | Dressing Applied | Dressing Applied | Dressing Applied | Dressing Applied |
| Time 3 hours | Weeping, Wet & Open | Dry and Closed | Dry and Closed | Dry and Closed |
| Time 6 hours | Wet, entirely wound | Dry, Closed flat | Dry, Closed, | Dry, Closed, |
| | indurated, | | induration limited to | induration limited to |
| | remains open | | edges of laceration | edges of laceration |
| Time 10 hours | Wet, reddened, | Dry, closed, flat, no | Dry, closed, flat, | Dry, closed, flat, |
| | indurated, open | induration | lessening of induration | lessening of induration |
| | | | at edges | at edges |
| Time 18 hours | Dry, reddened, | | | |
| | indurated, closing | | | |

Following Shower with wounds opened to water directly

| | | | | |
|---|---|---|---|---|
| Time 24 | Dry, reddened, | Dry, closed, flat, | Dry, partially opened | Dry, partially opened |
| hours | indurated, tender, | minimal induration | in center of wound, no | in center of wound, no |
| | partially opened the | | induration, with | induration, with |
| | entire length of wound | | hardened non- | hardened non- |
| | | | scabbing base of | scabbing base of |
| | | | wound covered, non- | wound covered, non- |
| | | | tender | tender |
| Day 2 | Dry, reddened, | Dry, closed, flat, no | Dry, partially opened, | Dry, partially opened, |
| | indurated, tender to | induration, non-tender | no induration, with | no induration, with |
| | touch, scab forming | | hardened non- | hardened non- |
| | | | scabbing base rising | scabbing base rising |
| | | | from bottom of wound | from bottom of wound |
| | | | toward top, non-tender | toward top, non-tender |
| Day 3 | Dry, completely | Dry, closed, flat, no | Dry, partially opened, | Dry, partially opened, |
| | scabbed, indurated, | induration, non-tender, | no induration, rising | no induration, rising |
| | tender to touch | shrinking in length | lining of base | lining of base |
| | | | extending to internal | extending to internal |
| | | | sides of wound, no | sides of wound, no |
| | | | tenderness | tenderness |
| Day 5 | Indurated, Peripheral | Completely healed | Wound with no | Wound with no |
| | edges of scab starting to | | induration, healed with | induration, healed with |
| | peel, with an adherent | | thin area of closing, no | thin area of closing, no |
| | tender central scab. | | tenderness | tenderness |
| Day 7 | Peripheral edges of scab | Completely healed | Completely healed | Completely healed |
| | peeled away exposing | | | |
| | 3 mm wide partially | | | |
| | healed undersurface, | | | |
| | with an adherent tender | | | |
| | central scab. | | | |

Each of the three embodiments of the invention tested, i.e., immune-modulating tetracycline, inulin, and fructo-oligosaccharde suspensions, as compared to the saline control, resulted in (i) accelerated healing, (ii) spontaneous wound closure and dryness within the first 3 hours of injury, (iii) less induration of the wound, (iv) no tenderness at any time during healing, and (v) completed healing by day 7. On the other hand, the wound treated with sterile sodium chloride solution persisted, with signs of inflammation and adherent scab formation still present at day 7.

### Example 9

### Patients with Irritable Bowel Syndrome (IBS) Treated with Immuno-Modulating Polysulfate Disaccharide Suspension

Sixty Bangladeshi patients randomized in a double-blinded placebo-controlled dyspepsia trial were treated for 28 days with either a ten percent suspension of immuno-modulating (IM) polysulfate disaccharide suspension twice daily (1.5 g of sucralfate per dose) or placebo consisting of the identical suspension devoid of sucralfate. Fifty patients completed the trial, 28 of whom had comorbid IBS, as defined by the Rome II classification.

Of these 28 IBS patients, ten were constipation dominant (cIBS), four of whom received placebo and six had received immuno-modulating (IM) polysulfate disaccharide suspension. Eighteen of the 28 were diarrhea dominant IBS (dIBS), eight of whom received placebo and ten had received immuno-modulating polysulfate disaccharide suspension. A number of patients treated with immune-modulating polysulfate disaccharide suspension reported improved boewl movements. Those with diarrhea reported less frequent stools that were not loose, and had no urgency or cramps. Those with constipation reported more frequent bowel movements, less bloating, and no pain. Of those receiving placebo, one of four with cIBS (25%) and two of eight with dIBS (25%) responded as just described. In contrast, among those treated with immuno-modulating polysulfate disaccharide, five of six with cIBS (83%) and eight of ten with dIBS (80%) responded as described.

Patients with Constipation Dominant IBS Treated with IM-Polysulfate vs. Placebo

| | Patients | Patients | Percent |
|---|---|---|---|
| Treatment | Treated | Responded | Response |
| Placebo | 4 | 1 | 25% |
| Immuno-modulating | 6 | 5 | 83% |
| polysulfate | | | |

Patients with Diarrhea Dominant IBS Treated with IM-Polysulfate vs. Placebo

| | Patients | Patients | Percent |
|---|---|---|---|
| Treatment | Treated | Responded | Response |
| Placebo | 8 | 2 | 25% |
| Immuno-modulating | 10 | 8 | 80% |
| polysulfate | | | |

### Example 10

### Patient with Constipative IBS Treated with Immuno-Modulating Polysulfate Disaccharide Powder

A 38 year old female diagnosed with slow transit constipation and IBS was constipated for 21 days. Despite the use of cathartics, laxatives, and stool softeners, she had managed to pass only small amounts of hardened stool. For two nights she took 2 capsules of immuno-modulating polysulfate disaccharide containing a total of 500 mg of sucralfate. On the third day she experienced a bowel movement, with relief of discomfort and bloating. She had regular bowel movements every 2 days as she continued to take 1-2 capsules nightly.

### Example 11

### Patient with Constipative IBS Treated with Immuno-Modulating Polygalacturonic-Rhamnose Suspension

A 47 year old woman diagnosed with cIBS using escalating doses of laxative to maintain regularity of movements without the sense of complete evacuation with movements. She had bowel movements every 4-5 days with incomplete partial moments every other day if aggressive with oral laxatives or enemas. Taking 60 ml of immuno-modulating polygalacturonic-rhamnose suspension, i.e., slippery elm complex, made in accordance to this invention the first day then 30 ml each evening before bed, she experienced a full bowel movement within 2 days. Continued administration of 30 ml of IM polygalacturonic-rhamnose suspension nightly resulted in regular movements once daily accompanied by the sense of complete evacuation.

### Example 12

### Patient with dIBS Treated with Immuno-Modulating Polygalacturonic-Rhamnose Powder

A 32 year old woman presented with a 12 year history of constipation alternating with urgent diarrhea, both associated with discomfort that can be partially relieved with bowel movements. She used a combination of laxative and anti-diarrheals to control her symptoms. Due to this stomach problem she never really felt well. She was diagnosed with IBS. After taking 2 capsules of immuno-modulating polygalacturonic-rhamnose powder containing a total of 600 mg of slippery elm complex, she experienced regularity of bowel movements within 3 days with no discomfort or bloating. As she continued on 1-2 capsules nightly, she had no recurrence of diarrhea or associated urgency.

### Example 13

### Use of Capsules Containing Immuno-Modulating Polysulfate Disaccharide in 129 Individuals with a Personal History of IBS

One hundred twenty nine individuals with a personal history of IBS requested and administered to themselves capsules of immuno-modulating polysulfate disaccharide (sucralfate complex), taking 2 capsules nightly for 2 nights followed by 1-2 capsules nightly. Among the 129 individuals, 51 were adult males and 78 were adult females. All individuals self-medicated for years with over-the-counter (OTC) laxatives and anti-diarrhea remedies with suboptimal results. All patients experienced an improvement of bowel movements beyond that achieved with OTC remedies following self-administration of the immuno-modulating polysulfate disaccharide of the current invention. Over seventy-five percent of the patients (40 of 51 males and 59 of 78 females) stopped taking OTC medications. Following 2 years of treatment, 3 males and 4 females stopped taking the capsules claiming that their irregular bowel habits were completely resolved and that they did not require OTC remedies. One year later, 2 of the 3 males resumed taking the capsules but at a reduced frequency of every 2-4 days with satisfactory restoration of bowel regularity without bloating and pain.

### Example 14

### Treatment of a Patient with Functional Dyspepsia Using Immuno-Modulating Polygalacturonic-Rhamnose Suspension

A 38 year old man diagnosed with functional dyspepsia according to the Rome II criteria suffered from post-prandial early satiety, bloating, and stomach indigestion. Administration of omeprazole, esomeprazole, pantoprazole, raberazole, and sucralfate suspension failed to improve his symptoms. Administration of 15 ml of a suspension of immuno-modulating polygalacturonic-rhamnose prior to each meal prevented the experience of early satiety, bloating and indigestion. Initially, there was only a lessening of post-prandial indigestion. Continued administration over a 1-2 week period led to a disappearance of functional dyspepsia altogether.

### Example 15

### Treatment of Non-Acidic Nocturnal Heartburn with Immuno-Modulating Sucralfate or Immuno-Modulating Polygalacturonic-Rhamnose Suspension

A 48 year old man was diagnosed with non-acidic nocturnal heartburn unresponsive to proton pump inhibitors or histamine-2 acid blockers. He experiences subxiphoid to substernal discomfort that causes him to wake between 1 am and 4 am 1-3 times per week. The discomfort transiently responds for 2-3 minutes to chewable calcium carbonate tablets.

The patient noted relief of discomfort while swallowing 30 ml of an immuno-modulating poly-sulfate suspension, with a complete cessation of discomfort by 5 minutes that lasted through the night. He experienced a similar response after swallowing the same dose of an immuno-modulating polygalacturonic-rhamnose suspension.

### Example 16

### Treatment of a Patient with Chemo-Radiation Ulcers and Enteritis Using Immuno-Modulating Polysulfate Suspension

Following surgery, a male patient with stage IVb head and neck squamous cell carcinoma underwent six weeks of chemotherapy and radiation including administration of paclitaxel, carboplatin, and treatment with 201 Gy of radiation. He received a gastrostomy feeding tube in anticipation of developing oral mucositis (OM) and alimentary mucositis (AM), which he did develop by the end of the second week of chemotherapy/radiation treatment. Administration of three daily doses of immuno-modulating polysulfate suspension (1.5 g per dose) initially reduced and then eliminated painful oral ulcerations, difficulty swallowing and loose stools. The patient remained free of ulcers, nausea, and loose stools, required no narcotic analgesia, and tolerated a normal diet without need of the gastrostomy tube feeding. His symptoms returned 1 week following cessation of treatment with immuno-modulating polysulfate suspension while receiving chemo-radiation, but abated within three days following resumption of the immuno-modulating polysulfate suspension.

### Example 17

### Treatment of a Patient with an Acute Skin Wound with Immuno-Modulating Polysulfate Suspension

A 16 year old male was diagnosed with a 2 cm by 0.4 cm beveled flap laceration to the middle phalanx of the right 4th finger. The wound was 0.3 cm deep. Typically, a wound of this type would require 10-12 days for complete healing if sutured and 21 days if simply bandaged and kept dry. His wound was treated with a bandage saturated with an immuno-modulating polysulfate suspension. The bandage was changed daily for 5 days. By day 6, complete healing was observed, far sooner than the 10 days normally required for healing of a sutured wound.

### Example 18

### Treatment of a Patient with Acute Skin Wounds with Immuno-Modulating Polygalacturonic-Rhamnose Suspension

A 25 year old male diagnosed with a 0.3 cm by 1 cm avulsion type laceration of the tip of the left thumb had his wound treated with a bandage saturated with an immuno-modulating polygalacturonic-rhamnose suspension. Initial scabbing of the laceration occurred in 2 days and the wound was completely healed in 6 days. A wound of this type and size typically requires 7 days before initial scabbing occurs and 16-20 days for complete healing.

### Example 19

### Accelerated Healing of Esophageal Erosions and Clinical Relief by Administering an Immuno-Modulating Polysulfate Disaccharide Suspension

Forty one patients having esophageal erosions were randomized to four treatment arms, with 39 patients (37 males and 2 females) completing the study. There were 11 patients receiving immuno-modulating polysulfate disaccharide suspension (average age 28.5 yrs; 1 dropped out), 10 in the omeprazole group (average age 28 yrs), 10 in the ranitidine group (average age 28.8 yrs; 1 dropped out) and 10 in the antacid group (average age 34.8 yrs). The trial period was seven days.

There were two primary end points, i.e., healing and no healing, and three secondary end points, i.e., no relief, partial relief, and complete relief of pain. Healing was defined as no evidence of erosions by endoscopy at day 8 that were present on day 0. No healing was defined as evidence of erosions remaining on day 8. No relief was defined as the persistence of symptoms regardless of severity; partial relief was defined as the loss of 1 or more symptoms and the persistence of those noted at day 0; and complete relief was defined as the loss of all symptoms by day 8 that had been present on day 0.

| | Number of | Complete | Complete | | |
|---|---|---|---|---|---|
| Treatment | Patients | Relief | Healing | Male | Female |
| Immuno-modulating | 10 (11) | 5 (50%) | 8 (80%) | 9 | 1 |
| polysulfate | | | | | |
| disaccharide | | | | | |
| suspension | | | | | |
| Omeprazole | 10 | 4 (40%) | 3 (30%) | 9 | 1 |
| H2B Ranitidine | 9 (10) | 3 (30%) | 0 (0%) | 9 | 0 |
| Antacid | 10 | 5 (50%) | 0 (0%) | 10 | 0 |
| (Magnesium | | | | | |
| Aluminum | | | | | |
| Hydroxide) | | | | | |

Eighty percent of patients who were administered an immuno-modulating polysulfate disaccharide suspension demonstrated complete healing of erosions within 7 days, far better than omeprazole (30%), ranitidine (0%) and antacids (0%). Fifty percent of patients treated with immuno-modulating polysulfate disaccharide suspension experienced complete symptomatic relief within 7 days, better than treatment with omeprazole (40%) or ranitidine (30%), and similar to antacids (50%).

### Example 20

### Treatment of Scours in Livestock with a Suspension of Immuno-Modulating Polysulfate and Immuno-Modulating Polygalacturonic-Rhamnose

A 3 week old calf diagnosed with scours (diarrhea) by a veterinarian was found to be dehydrated and required aggressive oral rehydration. Administration of bismuth-containing compounds failed to stop the diarrhea which worsened by the third day of treatment. Oral administration of 10 ml of an immuno-modulating polysulfate suspension three times daily stopped the diarrhea within 24-36 hours. The calf resumed suckling and grazing by day 4 while continuing the treatment with immuno-modulating polysulfate suspension.

A 5 week old weanling pig also diagnosed with scours by a veterinarian was found to have non-bloody diarrhea, dehydration, and poor appetite. The pig was treated orally with 5 ml of an immuno-modulating polygalacturonic-rhamnose suspension three times daily. This treatment resulted in the cessation of diarrhea within 36-48 hours.

### Example 21

### Treatment of Canine Parvo Enteritis with Immuno-Modulating Polysulfate Disaccharide Suspension

During an endemic outbreak of parvo in the community, a 1 year old 30 lb canine puppy presented with 4-5 daily episodes of vomiting and 3-4 daily bouts of non-bloody diarrhea and was diagnosed with parvo-enteritis by the veterinarian. The puppy was treated with 8 ml of immuno-modulating polysulfate suspension twice daily, resulting in relief of vomiting episodes within 12 hours and cessation of diarrhea within 24-36 hours. By day 3 the puppy's appetite had returned.

### Example 22

### Treatment of Canine Hemorrhagic Gastroenteritis with Immuno-Modulating Polygalacturonic-Rhamnose Suspension

A 4 year old mixed breed canine presenting with high fever, elevated white blood cell count, bloody diarrhea, anorexia, and dehydration was diagnosed with hemorrhagic gastroenteritis by a licensed veterinarian. This case was severe, and if the dog was to survive, it would take 5-7 days of hospitalization, intravenous hydration, antibiotics, tube feeding, and 8-9 days before a regular diet could be re-introduced. The animal was hydrated and treated intravenously with antibiotics. Oral administration of 10 ml of immuno-modulating polygalacturonic-rhamnose suspension every 8 hours resulted in cessation of diarrhea in 48 hours and return of appetite in 72 hours.

### Example 23

### Treatment of Equine Right Dorsal Colitis with Immuno-Modulating Polygalacturonic-Rhamnose Suspension

An 8 year old quarter horse diagnosed with right dorsal colitis by an equine veterinarian had been on high doses of bananime, a non-steroidal anti-inflammatory (NSAID) pain reliever. Right dorsal colitis is known to be caused by ulcerations within the colon of the horse. Treatment was initiated by cessation of the NSAID and administration of 8-12 grams sucralfate four times daily. The horse worsened after 2 days and required intravenous hydration. Oral administration to the horse of 30 ml of an immuno-modulating polygalacturonic-rhamnose suspension every 8 hours led to an improvement in attitude and appetite within 24 hours, a complete cessation of diarrhea in 36 hours, and the resumption of a regular diet in 72 hours.

### Example 24

### Treatment of Equine Ulcer Colic with Immuno-Modulating Polygalacturonic-Rhamnose Suspension

A 3 week old equine foal was diagnosed by a large animal veterinarian with gastroduodenal ulceration following 3 days of poor appetite. The foal's condition worsened rapidly, and by day 5, the foal was completely anorexic and demonstrated signs of colicky abdominal discomfort and diarrhea. Treatment with a proton-pump inhibitor and plain sucralfate failed to alleviate the symptoms. Oral administration of 10 ml of immuno-modulating polygalacturonic-rhamnose suspension every 8 hours resulted in the disappearance of signs of colicky abdominal discomfort within 36 hours, the tolerance of a light grain diet within 48 hours, and cessation of diarrhea after 1 day of treatment. The foal resumed normal diet and activity by day 4 of treatment.

### Example 25

### Accelerated Healing of Equine Ulcer with Immuno-Modulating Polygalacturonic-Rhamnose Suspension

An open-label field test of treatments for ulcer colic in horses was conducted. Of the 209 horses in the study, 173 failed to respond to standard treatment regimens including a combination of proton pump inhibitor (omeprazole), histamine 2 blocker (ranitidine or cimetidine), or antacids. Twenty-three of the non-responder horses were found to have ulcers by endoscopy. 8 of the non-responders were then treated with an immuno-modulating polygalacturonic-rhamnose suspension (30 ml 3 times daily for 1 day then twice daily) while the other 15 were treated with omeprazole, ranitidine, or antacids, together with an immuno-modulating polygalacturonic-rhamnose suspension. FDA approved omeprazole (GastroGard®) typically heals ulcers in 70% of subjects after 28 days of treatment (NDA 141-123, Mar. 16, 1999). Ulcers in all 23 horses receiving immuno-modulating polygalacturonic-rhamnose suspension were completely healed following 14-20 days of treatment. This represents an 8-14 day acceleration of healing as compared to the typical 28 days for GastroGard® treatment. Of note, this acceleration of healing occurred with ulcers in horses which were on immuno-modulating polygalacturonic-rhamnose suspension alone.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. An immuno-modulating composition for engaging a homeostasis-maintaining factor, the composition comprising an organic compound and a metal chelator or a metal ion, wherein a valency molar ratio between the organic compound and the metal chelator or metal ion is 1:1 to 10:1 and the organic compound contains covalently attached polar constituents such that the polar constituents have a minimally restrictive stereographic ability of rotation.

2. The immuno-modulating composition of claim 1, wherein the composition includes the organic compound, the metal ion, and the metal chelator, and the valency molar ratio between the organic compound, the metal ion, and the metal chelator is 1:1:1 to 10:1:1.

3. The immuno-modulating composition of claim 2, wherein the organic compound is selected from the group consisting of a fatty acid, a gum, a starch, a polyhydrol, a sulfonate, a phosphonate, a heterocyclic compound, a multi-cyclic compound, a carboxylic acid, and a carbamyl compound.

4. The immuno-modulating composition of claim 2, wherein the metal chelator is selected from the group consisting of a carboxylic acid, a heterocyclic compound, a multi-phenyl compound, an organic diamine, a polyphosphonate, mesalamine, a short, medium, or long chain fatty acid, gabapentin, and a carboxylate.

5. A method for treating a disease or condition in a subject, the method comprising administering to a subject in need thereof a therapeutically effective amount of a pharmaceutically active immuno-modulator that includes an organic compound and a metal chelator, wherein the valency molar ratio between the organic compound and the metal chelator is 1:1 to 10:1 and the organic compound contains covalently attached polar constituents such that the polar constituents have a minimally restrictive stereographic ability of rotation.

6. The method of claim 5, wherein the pharmaceutically active immuno-modulator further includes a metal ion and the valency molar ratio between the organic compound, the metal ion, and the metal chelator is 1:1:1 to 10:1:1; the organic compound is selected from the group consisting of a uni or polysulfate mono or polydisaccharide, polygalacturonic rhamnose, a fructo-oligosaccharide, and inulin; the metal ion is Ca²⁺ or Mg²⁺; and the metal chelator is malate, oxalate, citrate, ethylenediaminetetraacetic acid, or diethylenetriaminepentaacetic acid.

7. The method of claim 5, wherein the disease or condition is selected from the group consisting of epithelial lacerations, wounds, burns, ulcers, chemo-radiation oral ulcerations, oral mucositis, alimentary mucositis, gastroesophageal reflux disorder and erosions, Barrett's esophagitis, non-erosive reflux disorder, gastritis, gastroenteritis, enteritis, enterocolitis, functional dyspepsia, celiac sprue, collagenous or lymphocytic colitis, chemo-radiation enteritis, Crohn's disease, irritable bowel syndrome (IBS), chemo-radiation colitis, ulcerative colitis, minimal colitis, atherosclerosis, chronic hepatitis, muscle strains, myositis, tendinoligamentous strains, tendonitis, fibrositis, fibrosis, fibromyalgia, osseous injuries, post-surgical tissue states, mechanical post-traumatic tissue states, urinary or biliary cystitis, prostatitis, prostatic hypertrophy, orchitis, pelvic inflammatory disease, scours, parvoenteritis, hemorrhagic gastroenteritis, veterinary peptic ulcer, and colitis.

8. The method of claim 6, wherein the disease or condition is selected from the group consisting of epithelial lacerations, wounds, burns, ulcers, chemo-radiation oral ulcerations, oral mucositis, alimentary mucositis, gastroesophageal reflux disorder and erosions, Barrett's esophagitis, non-erosive reflux disorder, gastritis, gastroenteritis, enteritis, enterocolitis, functional dyspepsia, celiac sprue, collagenous or lymphocytic colitis, chemo-radiation enteritis, Crohn's disease, irritable bowel syndrome (IBS), chemo-radiation colitis, ulcerative colitis, minimal colitis, atherosclerosis, chronic hepatitis, muscle strains, myositis, tendinoligamentous strains, tendonitis, fibrositis, fibrosis, fibromyalgia, osseous injuries, post-surgical tissue states, mechanical post-traumatic tissue states, urinary or biliary cystitis, prostatitis, prostatic hypertrophy, orchitis, pelvic inflammatory disease, scours, parvoenteritis, hemorrhagic gastroenteritis, veterinary peptic ulcer, and colitis.

9. The method of claim 7, wherein the disease or condition is constipation dominant IBS, diarrhea dominant IBS, or mixed, alternating, and undifferentiated IBS.

10. The method of claim 5, wherein the pharmaceutically active immuno-modulator further includes a metal ion and the valency molar ratio between the organic compound, the metal ion, and the metal chelator is 1:1:1 to 10:1:1 and the organic molecule is selected from the group consisting of tetracycline, doxycycline, a polysulfate, a polyphosphonate, a fluoroquinolone, cefditoren pivoxil, rifaximin, benzydamine, cefuroxime axetil, and ezetimibe, or a derivative thereof covalently modified with one or more of a phosphate group, a sulfate group, and a nitrate group.

11. The method of claim 10, wherein the disease or condition is an epithelial laceration, the organic compound is tetracycline, the metal ion is Ca²⁺, and the metal chelator is malate, oxalate, citrate, ethylenediaminetetraacetic acid, or diethylenetriaminepentaacetic acid.

12. A method for delivering a growth factor or a homeostasis-maintaining factor to its receptor, the method comprising combining the growth factor or the homeostasis-maintaining factor with a composition that includes: (i) a polyionic carrier or an organic compound and (ii) a metal ion or a metal chelator, and contacting the combined growth factor or homeostasis-maintaining factor with a target cell, wherein the target cell contains a receptor for the growth factor or homeostasis-maintaining factor and the composition accelerates binding of the growth factor or homeostasis-maintaining factor to the receptor on the target cell and activation of the receptor as compared to the growth factor or homeostasis-maintaining factor alone.

13. The method of claim 12, wherein the composition includes the polyionic carrier and the polyionic carrier is selected from the group consisting of a fatty acid, a gum, a starch, a polyhydrol, a sulfonate, a phosphonate, a heterocyclic compound, a multi-cyclic compound, a carboxylic acid, and a carbamyl compound.

14. The method of claim 12, wherein the composition includes the organic compound and the organic compound is selected from the group consisting of a uni or polysulfate mono or polydisaccharide, polygalacturonic rhamnose, a fructo-oligosaccharide, inulin, tannin, resveratrol, a mucilage, a gum, a bile acid, and a starch, or a derivative thereof covalently modified with one or more of a phosphate group, a sulfate group, and a nitrate group.

15. The method of claim 12, wherein the composition includes the organic compound and the organic compound is selected from the group consisting of tetracycline, doxycycline, a polysulfate, a polyphosphonate, a fluoroquinolone, cefditoren pivoxil, rifaximin, benzydamine, cefuroxime axetil, and ezetimibe, or a derivative thereof covalently modified with one or more of a phosphate group, a sulfate group, and a nitrate group.

16. The method of claim 13, wherein the composition includes the metal ion and the metal chelator.

17. The method of claim 14, wherein the composition includes the metal ion and the metal chelator.

18. The method of claim 15, wherein the composition includes the metal ion and the metal chelator.
